SEQUENCE LISTING (6 sequences)

SEQ ID NO: 1 - DNA, 19 bp, Artificial Sequence
  Synthetic transposon terminal sequence
ctgtctcttatacacatct

SEQ ID NO: 2 - DNA, 19 bp, Artificial Sequence
  Synthetic transposon terminal sequence
agatgtgtataagagacag

SEQ ID NO: 3 - DNA, 72 bp, Artificial Sequence
  Synthetic Insert DNA F
ctgtctcttatacacatcttccgagcccacgagacuuaatcgtcggcagcgtcagatgtgtataagagacag

SEQ ID NO: 4 - DNA, 72 bp, Artificial Sequence
  Synthetic Insert DNA R
ctgtctcttatacacatctgacgctgccgacgauuaagtctcgtgggctcggaagatgtgtataagagacag

SEQ ID NO: 5 - DNA, 51 bp, Artificial Sequence
  Synthetic Tn5 Primer F
aatgatacggcgaccaccgagatctacactagatcgctcgtcggcagcgtc

SEQ ID NO: 6 - DNA, 48 bp, Artificial Sequence
  Synthetic Tn5 Primer R
caagcagaagacggcatacgagattcgccttagtctcgtgggctcgga

(19) United States
(12) Patent Application Publication
(10) Pub. No.: US 2020/0123536 A1
(43) Pub. Date: Apr. 23, 2020

(54) CIRCULAR TRANSPOSON COMPOUND AND APPLICATION THEREOF

(71) Applicant: BEIJING TRANSGEN BIOTECH CO., LTD., Beijing (CN)

(72) Inventors: Liang GENG, Beijing (CN); Zhen ZHU, Beijing (CN); Wen XIN, Beijing (CN)

(73) Assignee: BEIJING TRANSGEN BIOTECH CO., LTD., Beijing (CN)

(21) Appl. No.: 16/476,342

(22) PCT Filed: May 16, 2017
(86) PCT No.: PCT/CN2017/084515

§ 371 Date: Jul. 8, 2019

(30) Foreign Application Priority Data
Jan. 9, 2017 (CN) 201710013203.8

(51) Int. Cl. C12N 15/10; C12N 9/12; C40B 50/06; C12Q 1/6806

(52) U.S. Cl. CPC C12N 15/1082; C12Q 1/6806; C40B 50/06; C12N 9/1241

(57) ABSTRACT

The present disclosure relates to a circular transposon compound, wherein the circular transposon compound includes a Tn5 transposase and an insert DNA; and the insert DNA contains transposon end sequences, two tag sequences, and a DNA sequence containing an enzymecutting site. The present disclosure further relates to an application of the circular transposon in DNA library construction. In the present disclosure, the method of constructing a DNA library by using the circular transposon compound has simple and convenient operations and high library construction efficiency, wherein the whole process of the library construction can be completed in only 80 minutes, and the required DNA amount is significantly reduced, with 1-50 ng of target DNAs being sufficient for the library construction, thereby promoting the wide application of the next-generation sequencing technology in the life science research and precise medical enterprise, which has great and profound significance.

### CROSS REFERENCE TO PRIOR APPLICATION

This application is a National Stage Patent Application of PCT International Patent Application No. PCT/CN2017/084515 (filed on May 16, 2017) under 35 U.S.C. § 371, which claims priority to Chinese Patent Application No. 201710013203.8 (filed on Jan. 9, 2017), which are all hereby incorporated by reference in their entirety.

### REFERENCE TO A SEQUENCE LISTING SUBMITTED VIA EFS-WEB

The content of the ASCII text file of the sequence listing named “2019-12-30_13-506-0001_SequenceListing”, which is 1.66 kb in size, was created on Dec. 30, 2019 and electronically submitted via EFS-Web on Dec. 30, 2019, is incorporated herein by reference in its entirety.

### TECHNICAL FIELD

The present invention relates to the field of molecular biology and genomics, and more specifically, to a circular transposon compound and an application thereof.

### BACKGROUND ART

As an important experimental technology, the DNA sequencing (DNA sequencing) has a wide application in biological researches. The DNA sequencing technology was reported soon after the discovery of the DNA double helix structure. Due to the complicated operation process at that time, the DNA sequencing technology thus cannot be implemented in large scale. Subsequently, in 1977, Sanger invented the landmark dideoxy chain-termination sequencing method (Sanger, F.; Nicklen, S.; Coulson, A. R. (1977), “DNA sequencing with chain-terminating inhibitors”, Proceedings of the National Academy of Sciences USA, 74 (12): 5463-5467), which, along with the subsequent constant improvements, had been a mainstream technology of DNA sequencing in a considerable period of time due to its simplicity and rapidity.

However, with the development of science, the traditional Sanger sequencing method cannot fully satisfy the research needs. Both the genomere sequencing on model organisms and the genome sequencing on some non-model organisms require a sequencing technology with a lower cost, a higher throughput, and a faster speed. The next-generation sequencing(Next-generation sequencing) technology comes into being. Compared with the traditional Sanger sequencing method, the next-generation sequencing technology enables the scientists to sequence the DNA and RNA more quickly and cheaply, thereby revolutionizing researches of the molecular biology and genomics (Quail, M. A., I. Kozarewa, F. Smith, A. Scally, P. J. Stephens, R. Durbin, H. Swerdlow, and D. J. Turner. A large genome center's improvements to the Illumina sequencing system. Nat. Methods 2008, 5:1005-1010).

The standard construction of a traditional library for the next-generation sequencing includes the following steps of: (i) fragmentation, (ii) end repair, (iii) performing 5′endphosphorylation, (iv) 3′ end dA-tailing for a connection to the sequencing adaptor, (v) connecting adaptors, (vi) enrichment of the product of which both ends thereof are successfully connected to the adaptor by polymerase chain reaction (PCR) (as shown in FIG. 1) (Steven R. Head, H. Kiyomi Komori, Sarah A. LaMere, Thomas Whisenant, Filip Van Nieuwerburgh, Daniel R. Salomon, and Phillip Ordoukhanian, Library construction for next-generation sequencing: Overviews and challenges. BioTechniques, 2014, 56: 61-77). This method includes too many steps and cumbersome operations, and thus is time-consuming and laborious. So some scholars make improvements to integrate steps (ii) to (v) into the same reaction system (M Neiman, S Sundling, H Grönberg, P Hall, K Czene, et al. Library preparation and multiplex capture for massive parallel sequencing applications made efficient and easy. Plos One 2012, 7(7):e48616-e48616). However, no matter whether the integration is performed or not, the Taq or exoKlenow DNA polymerase is required for adding adA overhang to the DNA3′ end in the step of 3′ end dA-tailing in the library construction process. And the efficiency of the step of 3′ end dA-tailing is relatively low which is directly determined that the low efficiency of successfully connecting the adaptors to both ends of the product, thereby greatly restricting the construction effect of the library.

Some scholars have made improvements to the above construction method for a library (Chinese invention patent: 201610537963.4; invention title: EFFICIENT DNA ADAPTOR CONNECTION METHOD), wherein an adaptor mixture having different end structures is used for a connection to an A-tailed product. The blunt end, the 3′ dT overhang, and the 3′ dG overhang in the adaptor mixture respectively correspond and connect to the blunt end, the 3'dA overhang, and the 3'dG overhang structures which the DNA molecule end may have after the A-tailing reaction. Therefore, regardless of the A-tailing efficiency and the end structure of the DNA molecule, there is a matching adaptor for a connection thereto, thereby ensuring a significant improvement to the efficiency of adding adaptors to both ends of the molecule, and effectively improving construction effect of the library. However, this method still has the disadvantages such as too many steps and cumbersome operations.

A Bhasin et al. found that the Tn5 Transposon can insert any double-stranded DNA into other DNA fragments (A Bhasin, I Y Goryshin, W S Reznikoff. Hairpin formation in Tn5 transposition. Journal of Biological Chemistry, 1999, 274(52):37021-9), wherein the only and necessary condition is that both ends of the double-stranded DNA has a specific sequence (MEDS) of 19 bp. Epicentre developed the kit applying the Tn5 Transposon to the first-generation sequencing method according to this principle (EZ-Tn5<KAN-2>Insertion Kit).

After that, Epicentre found that the Transposon can be inserted to two different DNAs, as long as one end of the two DNA strands has the MEDS sequence. And based on this, he developed the second-generation DNA sequencing kit series products (TRANSPOSON END COMPOSITIONS AND METHODS FOR MODIFYING NUCLEIC ACIDS. United States Patent Application Pub. No. US 20110287435 A1.). Compared with the above-described construction solution of library, this method has more simple steps and is easier to operate, thereby significantly shortening the operation time of the library construction.

However, this method also has disadvantages: the method can theoretically add different tags to both ends of the DNA fragment formed by the breakage. But in the actual operation process, the direction of inserting the transposon into the sequence is random, which has two possibilities of positive and negative directions. Therefore, both ends of the DNA fragment may have the same tag which lead to the downstream PCR amplification thus cannot be performed. Actually, only 50% of the DNA fragments can be effectively utilized.

Therefore, it is necessary to develop a simple and efficient transposon complex and construction technology of DNA library which are easy to operate and have relatively low costs, and this is crucial for the life science research and precise medical enterprise.

### SUMMARY OF THE INVENTION

The first objective of the present invention is to provide a circular transposon compound, wherein the circular transposon compound can be used for genome DNA library construction and transcriptome sequencing library construction of the sequencing technology.

The second objective of the present invention is to provide an application of the above-described circular transposon compound.

The third objective of the present invention is to provide a construction method of DNA library, which completes the whole process of “fragmentation-tagging” in one step by performing “inserting and then cutting-off”, so as to improve construction efficiency of library and make operations more simple and convenient.

In order to achieve the above objectives, the present invention adopts the following technical solution:

a circular transposon compound, wherein the circular transposon compound comprises a Tn5 transposase and an insert DNA.

The insert DNA described in the present invention comprises transposon end sequences, two tag sequences, and a DNA sequence containing an enzymecutting site; the transposon end sequences are located at both ends of the insert DNA, the two tag sequences are located at the inner side of the transposon end sequences; the DNA sequence containing an enzyme cutting site is located between the two tag sequences; and the DNA sequence containing an enzymecutting site comprises but is not limited to a single-strand or double-stranded DNA sequence containing U, a DNA sequence containing a restriction enzymecutting site, or a double-stranded DNA sequence containing a non-complementary base pair.

In a preferred embodiment of the present invention, the circular transposon compound has only one insert DNA.

The insert DNA described in the present invention can be obtained by means of chemical synthesis or other molecular biological methods, and the structure thereof is as follows: “transposon end sequence-first tagsequence-enzymolysis sequence-second tag sequence-transposon end sequence”, wherein the first tag sequence and the second tag sequence can be identical or different, the constitution sequence is fixed, and each transposon is ensured to have only one insert DNA molecule. Therefore, when the two tag sequences are different, each transposon of such the structure can provide two completely precise different tag sequences, ensuring that adaptors at both ends of a breakage fragment are different, so as to maximize the utilization rate of a target DNA.

The present invention further provides an application of the circular transposon compound in DNA library construction.

The term “tag sequence” used in the present invention refers to a DNA sequence of a non-target nucleic acid component that provides an addressing means for a nucleic acid fragment connected thereto. In the present invention, a large number of DNA fragments are obtained after the target DNA is subjected to in-vitro transposition and enzymolysis processing, and tag sequences are introduced to both ends thereof. The tag sequence of the present invention can be flexibly and diversely designed according to different experimental requirements, so as to significantly expand an application range of the circular transposon compound of the present invention. For example, the tag sequence can be a PCR primer recognition sequence, a next-generation sequencing adaptor sequence (comprising a sequencer anchor sequence and asequencing primer recognition sequence) and the like, which can be used for genomeDNA library construction, transcriptome sequencing library construction, metagenomesequencing library construction, PCR fragment library construction, large-scale parallel DNA sequencing library construction, and the like of the next-generation sequencing technology. Therefore, all of the applications of the circular transposon compound of the present invention in the genomeDNA library construction, transcriptome sequencing library construction, metagenomesequencing library construction, PCR fragment library construction, large-scale parallel DNA sequencing library construction, and the like of the next-generation sequencing technology fall within the protection scope of the present invention.

The present invention further provides an efficient construction method of DNA library, comprising the following steps: obtaining a target DNA; preparing a circular transposon compound, the circular transposon compound comprising a Tn5 transposase and an insert DNA; and incubating the target DNA and the circular transposon compound, performing an enzymatic hydrolysis reaction, to obtain a DNA library. The insert DNA described in the present invention contains transposon end sequences, two tag sequences, and a DNA sequence containing an enzymecutting site; the transposon end sequences are located at both ends of the insert DNA, the two tag sequences are located at the inner side of the transposon end sequences; the DNA sequence containing an enzyme cutting site is located between the two tag sequences. The DNA sequence containing an enzymecutting site comprises but not limited to a single-strand or double-stranded DNA sequence containing U, a DNA sequence containing a restriction enzymecutting site, or a double-stranded DNA sequence containing a non-complementary base pair.

In the present invention, a corresponding enzyme is used for the enzymatic hydrolysis reaction according to different DNA sequences containing an enzymecutting site between the two tag sequences:

when there is a DNA sequence containing a restriction enzymecutting site between the two tag sequences, a restriction endonuclease is added for the enzymatic hydrolysis reaction;
when the DNA sequence between the two tag sequences is a single-stranded or double-stranded DNA sequence containing U, a UDGenzyme is added for the enzymatic hydrolysis reaction; and
when the DNA sequence between the two tag sequences is a double-stranded DNA sequence containing a non-complementary base pair, an exonucleasesuch as T7 endonuclease I, T4 endonuclease VII, or E. coli endonuclease V is added for the enzymatic hydrolysis reaction.

In the present invention, the Tn5 transposase and the insert DNA comprising the transposon end sequence (MEDS) at both ends and an enzyme cutting site located at middle are used to form the circular transposon compound, which is incubated with the target DNA (genomeDNA). The insert DNA is randomly inserted into the target DNA, in which case the target DNA does not break. Then a corresponding enzyme is used to process the target DNA, so as to widely break the target DNA. Therefore, when the insert DNA carries two different tag sequences, the target DNA fragment after the breakage whose tag sequences at both 5′ and 3′ ends are derived from the insert DNA has becomes a DNA fragment carrying different tag sequences at both ends. By designing the tag sequence in the inserted DNA, a large number of DNA fragments carrying different tag sequences at both ends can be harvested by means of a combined action of the Tn5 transposase and the enzyme capable of breaking the enzyme cutting site. Then, the construction work of library using the target DNA as a sequencing target can be completed by using these fragments as templates in combination with an amplificative step such as the PCR.

The present invention discloses a technology of breaking the target DNA and introducing a tag sequence to the end of the DNA fragment formed by the breakage through the combination of circular transposon and the enzymatic hydrolysis reaction. The size distribution of the tagged DNA fragments generated by the breakage can be simply controlled by adjusting the concentration of the circular transposon and the target DNA in the reaction system and the specific conditions of the in-vitro transposition reaction.

In a preferred embodiment of the present invention, the circular transposon compound can be purified after being formed, to remove the Tn5 transposase and the insert DNA which are not involved in the reaction and obtain a pure circular transposon, thereby improving the transposition efficiency and stabilizing the experimental result.

In a preferred embodiment of the present invention, the UDG enzyme is used to breakanin-vitro transposition product; a reaction condition of the UDG enzyme is very broad, so the UDG enzyme can be first used alone to process the transposition product or can act in combination with a DNA polymerase (for example, a PCR enzyme) in the same reaction system, so as to complete the whole process of “fragmentation-tagging-amplification” in one step.

Further, the circular transposon compound described in the present invention has one and only one insert DNA.

The beneficial effects of the present invention are as follows:

The traditional construction method of sequencing library has too many steps and cumbersome operations and is time-consuming and laborious. And the library construction efficiency is low which would take at least 4 hours to complete the library construction and require a large amount of DNAs, usually 1-5 μg of DNAs being required for library construction templates. Neiman et al. integrated the steps of the method and simplified the operations to some extent after the improvement, but it still takes about 3 hours to complete the library construction, and the required DNA amount is 5-1000 ng, which is still relatively high. In addition, the above library construction method is improved based on the Neiman method by using an adaptor mixture having different end structures for a connection to the A-tailed product, so as to improve the library construction efficiency by improving the adaptor connection efficiency, but simplification of the operation process of the library construction is limited.

In the present invention, the method of constructing a library by using the circular transposon compound has simple and convenient operations and high library construction efficiency, wherein the whole process of the library construction can be completed in only 80 minutes, and the required DNA amount is significantly reduced, with 1-50 ng of target DNAs being sufficient for the library construction, and the cost of the method is greatly lower than the commercial kits of companies such as Illumina, thereby promoting the wide application of the sequencing technology in the life science research and precise medical enterprise, which has great and profound significance.

### DESCRIPTION OF THE DRAWINGS

The specific embodiments of the present invention will be described in further detail below with reference to the drawings.

FIG. 1 illustrates a schematic diagram of a traditional construction principle of next-generation sequencing library;

FIG. 2 illustrates a schematic diagram of a principle of the present invention;

FIG. 3 illustrates a schematic diagram of library construction by PCR technology using a breakage fragment of the present invention;

FIG. 4 illustrates an electrophoretogram of a reaction product form by a transposons and a target DNA in Embodiment 1 processed by the Agilent high-sensitive DNA chip;

FIG. 5 illustrates a structure of a DNA fragment in a next-generation sequencing library successfully constructed in Embodiment 1; and

FIG. 6 illustrates an electrophoretogram of a PCR reaction template and product in Embodiment 1 processed by the Agilent high-sensitive DNA chip.

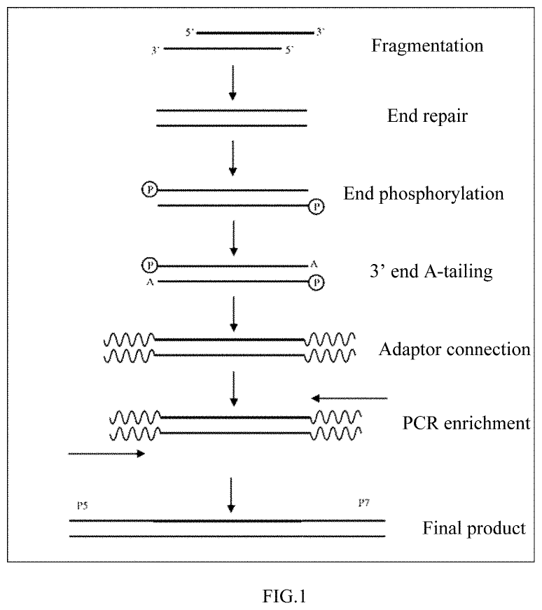

### DETAILED DESCRIPTION OF EMBODIMENTS

In order to describe the present invention more clearly, the present invention will be further described below with reference to the preferred embodiments and the drawings. One skilled in the art should understand that the following detailed description is merely for illustration instead of limitation, and the protection scope of the present invention should not be limited thereto.

### Materials Used in the Embodiments and Sources Thereof

UDG reaction mixture, MagicPure™ Size Selection DNA Beads, and TransNGS™ Library Amplification SuperMix(2×) (Beijing TransGen Biotech Co., Ltd.);
Agilent 2100 high-sensitive DNA chip (Agilent Inc.);
Qubit high-sensitive DNA test reagent (Thermo Fisher Scientific Inc.);
DNA synthesis (Life technologies Inc.); and
next-generation sequencing (Beijing Novogene Biological Information Technology Co., Ltd.).

### Embodiment 1 Verification as to Whether the Circular Transposon Compound is Capable of Achieving Both Breakage of a Target DNA and Insertion of a Tag Sequence

### 1. Preparation of an Insert DNA

Two single-strand DNA sequence of a length of 72 nt and carrying transposon end sequences (a sequence including a phosphorylated 5′ is as shown in SEQ ID No. 1, and a sequence including 3′ is as shown in SEQ ID No. 2) at both ends are synthesized, wherein the inner side of the transposon end sequences carries two different tag sequences (the tag sequences are underlined), and two Us are located between two different tag sequences, and the sequences are as follows:

Insert DNA F:
5′-CTGTCTCTTATACACATCTTCCGAGCCCACGAGACUUAATCGTCGGCA
GCGTCAGATGTGTATAAGAGACAG-3′ (as shown in SEQ ID No.
3 of the sequence table)
Insert DNA R:
5′-CTGTCTCTTATACACATCTGACGCTGCCGACGAUUAAGTCTCGTGGGC
TCGGAAGATGTGTATAAGAGACAG-3′ (as shown in SEQ ID No.
4 of the sequence table)

The powders of the two synthesized single-stranded DNAs are respectively dissolved by using an 1×hybridisation buffer (10 mM Tris-HCl pH8.0, and 50 mM NaCl), with a concentration of 200 μM, and an annealing reaction is performed after equal-volume mixing. The conditions of annealed product are as follows: 95° C., 5 minutes, slowly cooling to room temperature. 1 μl of the product is used for electrophoresis and detection in 2% agarose gel.

The annealed product is the insert DNA, which is a double-stranded DNA including 72 bp with the concentration of 100 μM. The structure thereof satisfies “transposon end sequence-first tag sequence-enzymolysis sequence-second tag sequence-transposon end sequence”.

### 2. Preparation of a Circular Transposon

A transposase Tn5 is quantified by means of the bicinchoninic acid (BCA) method, and the molar concentration thereof is calculated.

A Tn5 stock solution is prepared as follows: 50 mM HEPES-KOH pH 7.2, 0.1 M NaCl, 0.1 mM EDTA, 1 mM DTT, 0.1% Triton X-100, and 10% glycerol.

A preparation of reaction system is as follows:

Tn5: (final concentration: 2 μM)×μl;
insert DNA (100 μM): (final concentration: 2 μM) 2 μl; and
Tn5 stock solution: increasing to 100 μl.
Reaction conditions are as follows: 30° C., 1 hour, and −20° C. for storage.

### 3. Reaction Between the Transposon and the Target DNA and UDG Enzymatic Hydrolysis Reaction (as Shown in FIG. 2)

50 ng of Escherichia coliO157:H7genomic DNA is used as the target DNA, the amount of the transposon is 0.5 μl/1 μl/2 μl, and a preparation of system of transposition reaction is as follows:

target DNA: 50 ng;
transposon: 0.5 μl/1 μl/2 μl;

### 50 mM TAPS-NaOH pH 8.5.25 mM MgCl₂: 6 μl; and

dd H₂O: increasing to 30 μl.

Reaction conditions are as follows: 55° C., 5 minutes; then adding 30 μl of UDG reaction mixture, 55° C., 5 minutes.

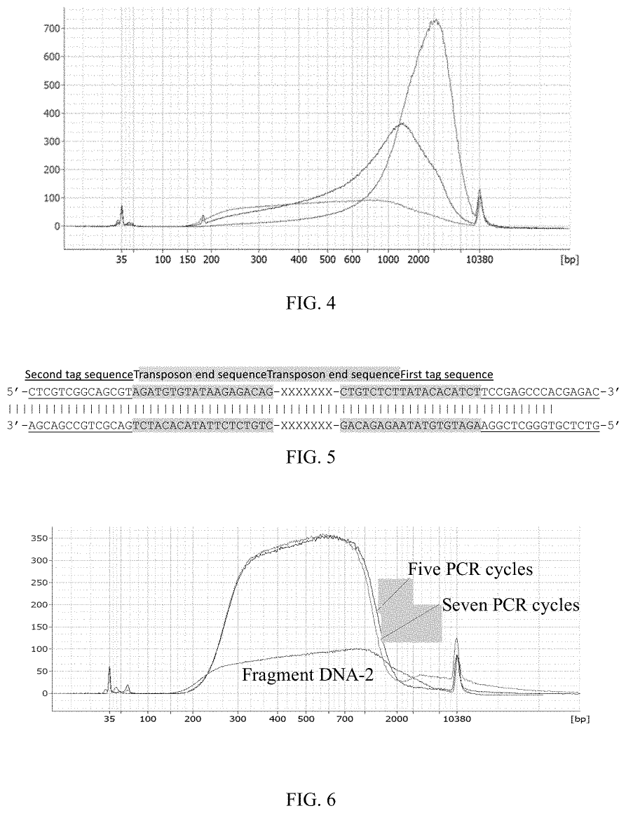

60 μl of the above reaction mixture is purified by using 1.0×MagicPure™ Size Selection DNA Beads. Product prepared by 0.5 μl of the transposon is named “fragment DNA-0.5”. Product prepared by 1 μl of the transposon is named “fragment DNA-1”. Product prepared by 2 μl of the transposon is named “fragment DNA-2”. And a fragment size of the fragment DNA is measured by using the Agilent 2100 high-sensitive DNA chip (as shown in FIG. 4).

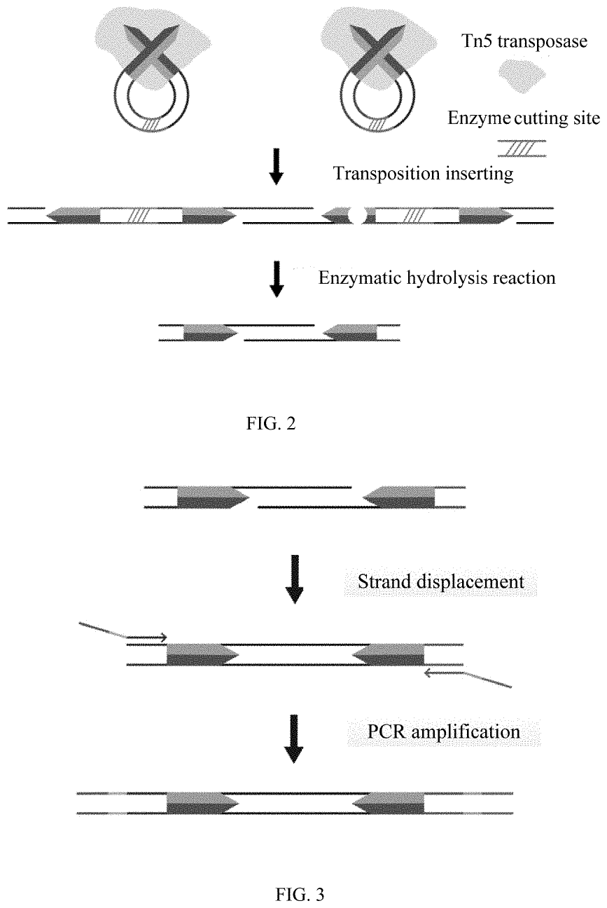

### 4. Verification of Reaction Product (a Reaction Principle is as Shown in FIG. 3)

The structure of a DNA sequence of product reacted successfully in a sequencing library is as shown in FIG. 5:

A specific primer is designed according to different tag sequences at both ends of the reaction product, the fragment DNA-2 is used as a template, and the PCR method is used for verifying whether the reaction is successfully performed. Primer sequences is as follows:

Tn5 Primer F:
5′-AATGATACGGCGACCACCGAGATCTACACTAGATCGCTCGTCGGCAG
CGTC-3′ (as shown in SEQ ID No. 5)
Tn5 Primer R:
5′-CAAGCAGAAGACGGCATACGAGATTCGCCTTAGTCTCGTGGGCTCGG
A-3′ (as shown in SEQ ID No. 6)

A PCR reaction system is as follows:

fragmentDNA-2: 20 μl;

### Tn5 Primer F (10 μM): 2.5 μl;

### Tn5 Primer R (10 μM): 2.5 μl; and

### TransNGS™ Library Amplification SuperMix(2×): 25 μl.

PCR reaction conditions are as follows:

72° C., end maturation, 3 minutes;
98° C., pre-denaturation, 3 minutes;
98° C., denaturation, 30 seconds;
62° C., anneal, 30 seconds;
72° C., extension, 30 seconds; and
72° C., post-extension, 3 minutes;
wherein the denaturation, anneal, and extension are performed for 5/7 cycles.

A PCR product is purified by using 1.0×MagicPure™ Size Selection DNA Beads, and a fragment size of the product is measured by using the Agilent 2100 high-sensitive DNA chip (as shown in FIG. 6). The concentration is measured by using the Qubit high-sensitive DNA test reagent, and the amount of PCR template and output are calculated, which are respectively as follows: a template amount of the fragment DNA-2 is totally 28 ng, and a production after 5 PCR amplifications is 370 ng and a production after 7 PCR amplifications is 1200 ng. The used primers are specific primers designed for tag sequences a and b, so it can be known that the transposon successfully achieve breakage of the target DNA while the tag sequences are inserted.

In addition, it can be known from the experiment that: a fragmentation degree of the target DNA can be simply controlled by adjusting a reaction ratio of the transposon to the target DNA. The reaction between the transposon and the target DNA and the UDG enzymatic hydrolysis reaction totally require 10 minutes and substitute for the fragmentation, end repair, 5′ end phosphorylation, 3′ end dA-tailing, and adaptor connection process of the traditional construction of next-generation sequencing library, thereby greatly simplify construction procedure of the next-generation library.

### Embodiment 2 Verification as to Whether the Method of the Present Invention can Serve as an Efficient Construction Method of Next-Generation Sequencing Library

### 1. Preparation of an Insert DNA

Refer to step 1 in Embodiment 1.

### 2. Preparation of a Circular Transposon

Refer to step 2 in Embodiment 1.

### 3. Reaction Between the Transposon and the Target DNA and UDG Enzymatic Hydrolysis Reaction

50 ng of human blood genomic DNA is used as the target DNA, and the amount of the transposon is 2 μl. The step is the same as step 3 in Embodiment 1.

### 4. PCR Amplification

Refer to step 4 in Embodiment 1, wherein the number of PCR cycles is 7.

### 5. Selection of Product Fragment Sizes by Using the Magnetic Bead Method

According to requirements of the sequencing company for the fragment size of a next-generation sequencing library, the fragment sizes are selected by using the MagicPure™ Size Selection DNA Beads, wherein a scale of magnetic bead of the first round is 0.6×, a scale of magnetic bead of the second round is 0.15×, the resultant product is the next-generation sequencing library, and the library is named “Tn5_Human”

### 6. Verification of a Library Quality by Using a Next-Generation Sequencer

The library is sequenced by the Illumina Hiseq X™ system, and a sequencing strategy is PE150. Quality control of sequencing data is as shown in Table 1, and comparison results between the sequencing data and a reference genomic sequence are as shown in Table 2.

TABLE 1
Quality control of sequencing data

| | Original | Effective | | | | | |
|---|---|---|---|---|---|---|---|
| | data | data | Effective | | | | GC |
| Library | output | amount | data rate | Base error | Q20 | Q30 | amount |
| name | (bp) | (bp) | (%) | rate (%) | (%) | (%) | (%) |
| Tn5_Human | 36,104,772,900 | 34,453,793,100 | 95.43 | 0.02 | 97.26 | 93.34 | 42.31 |

TABLE 2
Comparison results between the sequencing
data and a reference genomic sequence

| Library | Comparison | Coverage | Repetition | Sequencing |
|---|---|---|---|---|
| name | rate (%) | rate (%) | rate (%) | depth |
| Tn5_Human | 99.37 | 98.99 | 15.00 | 9.88 |

It can be seen from table 2 that: for human genomic DNA with a relatively large genome, a coverage rate of 98.99% can be achieved by using an initial sample amount of just 50 ng in the situation of a sequencing depth is about 10×, a effect of construction method of the next-generation sequencing library can rival that of construction method of the traditional library. And the construction process of library is fast, the operation is simple, and required sample amount is less.

Apparently, the above-described embodiments of the present invention are merely illustrations for clear description of the present invention instead of limitations to the implementation manners of the present invention, one skilled in the art can further make other variations or modifications of different forms on the basis of the above description, all of the implementation manners cannot be listed herein, and any obvious variations or modifications derived from the technical solution of the present invention still fall within the protection scope of the present invention.

1. A circular transposon compound, wherein the circular transposon compound comprises a Tn5 transposase and an insert deoxyribonucleic acid (DNA).

2. The circular transposon compound according to claim 1, wherein the insert DNA contains transposon end sequences, two tag sequences, and a DNA sequence containing an enzyme cutting site; the transposon end sequences are located at both ends of the insert DNA, the two tag sequences are located at the inner side of the transposon end sequences; and the DNA sequence containing an enzyme cutting site is located between the two tag sequences.

3. The circular transposon compound according to claim 2, wherein the DNA sequence containing an enzyme cutting site is a single-strand or double-stranded DNA sequence containing U, or a DNA sequence containing a restriction enzyme cutting site, or a double-stranded DNA sequence containing a non-complementary base pair.

4. The circular transposon compound according to claim 1, wherein the circular transposon has only one insert DNA.

5. An application of the circular transposon compound according to claim 1 in construction of DNA library.

6. A construction method of DNA library, wherein the method comprises: obtaining a target DNA; preparing a circular transposon compound, the circular transposon compound comprising a Tn5 transposase and an insert DNA; incubating the target DNA and the circular transposon compound; and performing an enzymatic hydrolysis reaction, to obtain a DNA library.

7. The construction method according to claim 6, wherein the insert DNA contains transposon end sequences, two tag sequences, and a DNA sequence containing an enzyme cutting site; the transposon end sequences are located at both ends of the insert DNA, the two tag sequences are located at the inner side of the transposon end sequences; and the DNA sequence containing an enzyme cutting site is located between the two tag sequences.

8. The construction method according to claim 7, wherein the DNA sequence containing an enzyme cutting site is a single-stranded or double-stranded DNA sequence containing U, or a DNA sequence containing a restriction enzyme cutting site, or a double-stranded DNA sequence containing a non-complementary base pair.

9. The construction method according to claim 8, wherein if the DNA sequence between the two tag sequences is a single-stranded or double-stranded DNA sequence containing U, a uracil-DNA glycosylase (UDG) enzyme is added for the enzymatic hydrolysis reaction; or
if there is a DNA sequence containing a restriction enzyme cutting site between the two tag sequences, a restriction endonuclease is added for the enzymatic hydrolysis reaction; or
if there is a double-stranded DNA sequence containing a non-complementary base pair between the two tag sequences, an exonuclease is added for the enzymatic hydrolysis reaction.

10. The construction method according to claim 6, wherein the circular transposon has only one insert DNA.

11. An application of the circular transposon compound according to claim 2 in construction of DNA library.

12. An application of the circular transposon compound according to claim 3 in construction of DNA library.

13. An application of the circular transposon compound according to claim 4 in construction of DNA library.
